(19) United States
(12) Patent Application Publication
(10) Pub. No.: US 2013/0211263 A1
(43) Pub. Date: Aug. 15, 2013

(54) INTUBATION GUIDE

(71) Applicant: THE BOARD OF REGENTS OF THE UNIVERSITY OF NEBRASKA, (US)

(72) Inventor: Benje Boedeker, Omaha, NE (US)

(21) Appl. No.: 13/799,775

(22) Filed: Mar. 13, 2013

Related U.S. Application Data
(63) Continuation-in-part of application No. 13/552,803, filed on Jul. 19, 2012.
Application No. 13/552,803 is a continuation of application No. 12/079,031, filed on Mar. 24, 2008, now abandoned.
(60) Provisional application No. 60/896,619, filed on Mar. 23, 2007.

(51) Int. Cl. A61M 16/04; A61B 1/04; A61B 5/00; A61B 1/267

(52) U.S. Cl. CPC A61M 16/0488; A61B 1/267; A61B 1/04; A61B 5/0084
USPC 600/478; 128/200.26; 600/188

(57) ABSTRACT

Apparatuses for guiding an endotracheal tube during intubation and associated methods of their use; the apparatuses formed to contain a plastic element able to hold deformations so as to conform the apparatus to the shape of a patient's endotracheal airway. The plastic element has sufficient give so as to minimize traumatic engagement with the inner surface of the airway lumen. Methods for using the apparatuses exploit indirect visualization where the person performing the intubation can shape the apparatuses so as to place the distal end into the field of view of the indirect visualization instrument even with minimal alignment of the patient's airway.

### CROSS REFERENCE TO RELATED APPLICATION

The present application is a Continuation-in-Part of U.S. application Ser. No. 13,552,803, filed Jul. 19, 2012 and titled “INTUBATION GUIDE,” which is a Continuation of U.S. application Ser. No. 12/079,031, filed Mar. 24, 2008 and titled “INTUBATION GUIDE,” now abandoned, which claims priority under 35 U.S.C. §119(e) to U.S. Provisional Application No. 60/896,619, filed Mar. 23, 2007 and titled “INTUBATION AND VENTILATION DEVICES;” which applications are herein incorporated by reference in their entirety.

### GOVERNMENT SUPPORT

This invention was made with government support under Grant No. W81xwh0610019 awarded by the Telemedicine and Advanced Technology Research Center. The government has certain rights in the invention.

### BACKGROUND

A secure airway is essential to manage anesthetized or critically ill patients. Maintaining ventilation through an endotracheal tube is a critical to maintaining a patient's airway.

Placing an endotracheal tube (endotracheal intubation or intubation), especially when done by those lacking extensive experience with the procedure, introduces substantial risk (See Field Airway Management Disasters 104 ANESTHESIA & ANALGESIA 482 (2007) herein incorporated by reference). Accidental placement of the endotracheal tube in the esophagus, for example, can kill a patient if not immediately detected. In instances of a difficult airway, intubation may not be possible even when performed by skilled anesthesiologist.

The factors that make an intubation difficult complicate the person performing the intubation (“the intubator”) ability to confirm the appropriate placement of the endotracheal tube. Confirmation of endotracheal tube placement is difficult given the anatomy of the patient's airway. In order to see the glottic opening, the ultimate destination of an endotracheal tube, the intubator must align the glottic opening with the mouth. Even after proper alignment multiple factors can obstruct visualization of the glottic opening resulting in a difficult airway.

A difficult airway is defined as the clinical situation where a clinically trained anesthesiologist experiences difficulty with face mask ventilation of the upper airway, difficulty with intubation, or both. Several factors can make for a difficult airway. Features of the patient, such as long upper incisors, a highly arched or narrow pallet, a low range of motion of the head and neck, or poor visibility of the uvula can indicate a difficult airway (See A clinical sign to predict difficult tracheal intubation; a prospective study 32 CANADIAN ANESTHETISTS' SOCIETY JOURNAL 429 (1985) herein incorporated by reference). The factors make for a difficult airway because they obstruct direct visualization of the glottic opening even when the intubator aligns the patient's airway.

The practice guidelines of the American Society of Anesthesiologists (See Practice Guidelines for Managing the Difficult Airway, 98 ANESTHESIOLOGY 1269 (2003) herein incorporated by reference) recognize multiple strategies to intubate a patient in the presence of a difficult airway. Utilizing a different laryngoscope blade, a laryngeal mask as an intubation conduit, fiber optic intubation, a light wand, retrograde intubation or use of an intubating stylet, bougie or a tube changer are all alternative strategies where traditional laryngoscopy cannot intubate when a difficult airway is present.

Among the initial steps in endotracheal intubation is alignment of the patient's airway (See Orotracheal Intubation, 356 N. ENG. J. MED e15 (2007) herein incorporated by reference). Similarly, the associated tools used to guide an endotracheal tube are linear and unable to conform to the natural shape of the patient's airway are inoperable in the absence of an aligned airway. To that extent, the potential of instruments that can indirectly visualize the glottic opening has yet to be realized. Such indirect visualization instruments, like video laryngoscopes, video laryngeal masks or fiber optic bougies can visualize the glottic opening even when the patient's airway is unaligned (a process herein called “indirect visualization”). Current tools used to guide the placement of an endotracheal tube, however, cannot fully exploit the potential of indirect visualization because these tools can only be used in the presence of an aligned airway.

Indirect visualization instruments can see ‘around the corner’ and visualize the glottic opening even when an airway is difficult or incompletely aligned. The present art currently offers no tools that can similarly reach ‘around the corner’. As a result, the present art cannot realize the benefits of indirect visualization instruments because they cannot operate in the field of vision provided by an indirect visualization instrument. The present invention teaches an apparatus that can be used, in conjunction with indirect visualization, which will fulfill the enormous potential of indirect visualization and shift the current alignment paradigm.

### SUMMARY

An apparatus is disclosed that can traverse the length of an endotracheal tube and comprises an element that conveys a degree of rigidity and the ability to undergo plastic deformation (a “plastic element”). Said plastic element having sufficient plasticity so as to retain a shape bent into it by an intubator during insertion into the patient's endotracheal airway but having sufficient give so as to minimize or avoid traumatic engagement with the inner surface of the airway lumen. The present disclosure teaches several embodiments of the plastic element: a ductile member received by the body of the endotracheal intubation guide, a characteristic of the material substantially comprising the endotracheal intubation guide or a member preformed to contain multiple bends conforming to the angles of a patient's unaligned airway.

The present disclosure further provides for the exploitation of the view of the glottic opening provided by indirect visualization instruments by teaching methods for utilizing the endotracheal intubation guide in conjunction with the indirect visualization instruments. Specifically, the present invention teaches methods for shaping, placing and confirming placement of the endotracheal intubation guide. The present invention further teaches methods for using the endotracheal intubation guide to place and confirm the placement of an endotracheal tube.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

### BRIEF DESCRIPTION OF THE DRAWINGS

The detailed description is described with reference to the accompanying figures. The use of the same reference numbers in different instances in the description and the figures may indicate similar or identical items.

FIG. 1A is an elevational view of an intubation guide apparatus undergoing plastic deformation. FIG. 1B is a diagrammatic depiction of a formed apparatus useful in intubation.

FIG. 2A is an elevational view of one embodiment undergoing plastic deformation. FIG. 2B cross sectional view of one embodiment and FIG. 2C is a longitudinal cross section of one embodiment.

FIG. 3A is a cutaway elevational view of an embodiment. FIG. 3B is an elevational view of one embodiment. FIG. 3C is an isometric view of one embodiment with parts broken away to reveal details of construction.

FIG. 4A depicts a formed intubation guide and FIG. 4B depicts deformation in avoidance of traumatic engagement with the inner wall of the airway lumen.

FIG. 5A is an elevational view depicting a pre-formed embodiment undergoing deformation and FIG. 5B is an elevational view depicting the elasticity of the apparatus returning to substantially its original form.

FIG. 6A is a cross section of the patient's endotracheal airway showing the positioning of a visualization instrument and an intubation guide.

FIG. 6B is a cross sectional view of the glottic opening and FIG. 6C is also a cross sectional view of the glottic opening.

### DETAILED DESCRIPTION

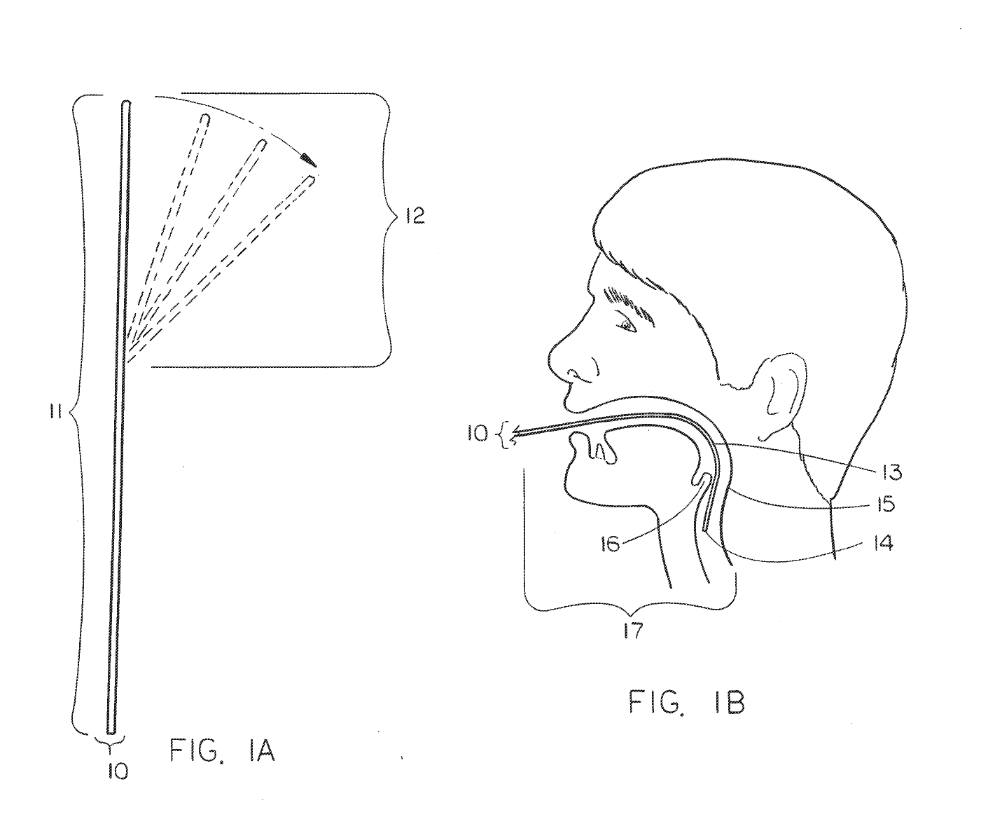

FIG. 1A illustrates an endotracheal intubation guide 10 that contains a plastic element 25 (not pictured) throughout all or substantially all of the body 11 in accordance with the present disclosure. The plastic element 25 is sufficiently malleable so as to enable the endotracheal intubation guide 10 to deform when bent 12 and sufficiently plastic so as to substantially maintain a shape bent 12 into it. The operator performing the intubation (“the intubator”) shapes the intubation guide 10 to substantially conform to the patient's endotracheal airway. The endotracheal intubation guide 10 and is formed to be a long, non-rigid, malleable member of sufficient length to traverse the length of the patient's endotracheal airway. To facilitate engagement with an endotracheal tube the member is generally round but could have an angular or other shape that can conform to the endotracheal tube lumen.

FIG. 1B depicts an endotracheal intubation guide 10 that has been shaped (a shaped endotracheal intubation guide 13). The shaped endotracheal intubation guide 13 is better able to conform to the natural shape of a patient's endotracheal airway. When inserting the intubation guide into the patient's airway, the intubator inserts a first end into the patient's airway, the distal end 14. The shaped endotracheal intubation guide 13 is shaped so that the distal end 14 can be introduced into the airway 41, around the epiglottis 16, and into the glottic opening 62 (not pictured). When shaped properly, the shaped endotracheal intubation guide 13 can be used to place the distal end into the glottic opening 62 (not pictured) when the patient's airway is un-aligned 17. The endotracheal intubation guide 10 must have sufficient give so that the plastic element 25 (not pictured) will deform before traumatically engaging the walls of the lumen of the airway.

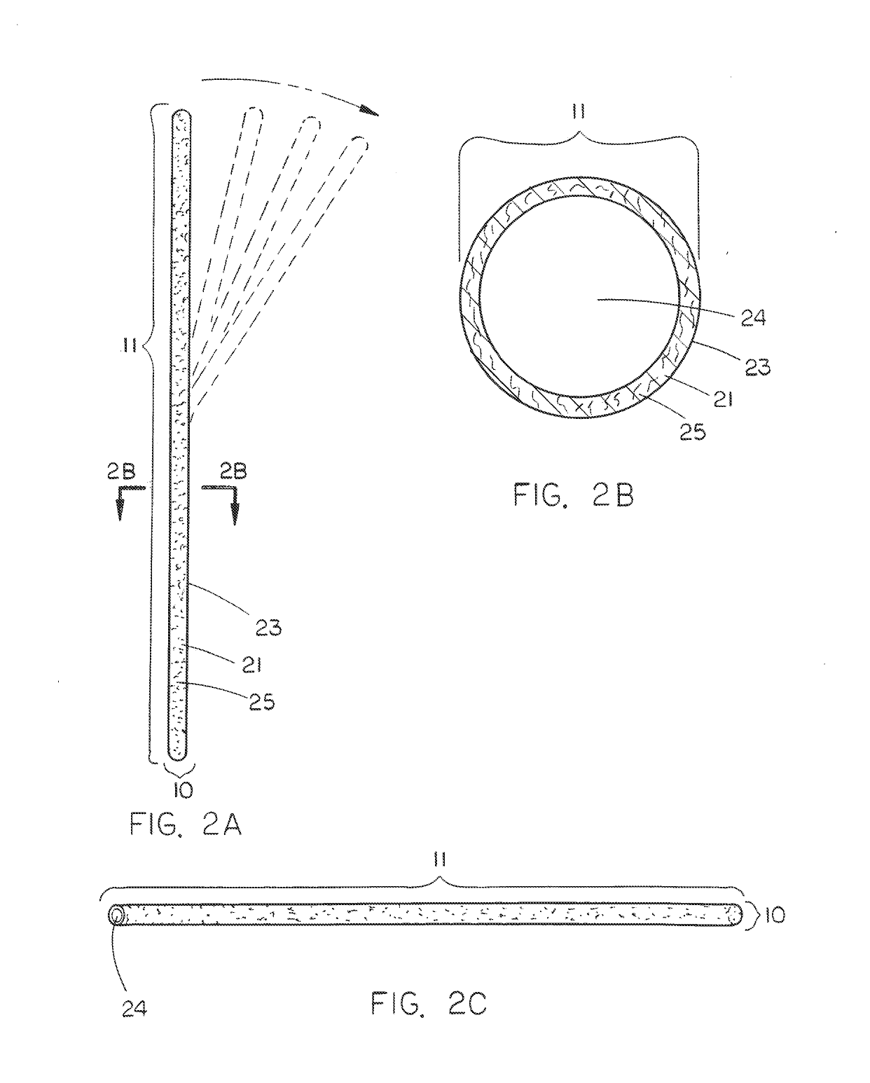

Looking now to FIG. 2A, in one embodiment, all or substantially all of the endotracheal intubation guide 10 is formed from a single material 21 that forms both the plastic element 25 of the endotracheal intubation guide 10 and all or substantially all of the body 11. The single material 21 can be any soft biocompatible material with sufficient plasticity or any blend of a variety of soft, biocompatible materials. Any variety of synthetic polymers, with the addition of appropriate plasticizers and co-polymers could form the single material 21. Polymers like polyethylene or polydiaxanone, for example, with the addition of one or more biocompatible plasticizers, such as polyethylene glycol, would have plasticity and malleability sufficient to practice the invention. Moreover, the addition of any variety of co-polymers would provide wide latitude to vary the plasticity, elasticity, give and durability of the single material 21 and, ultimately, the endotracheal intubation guide 10.

In embodiments where the single material 21 is comprised primarily of synthetic polymers, the single material 21 could be formed of woven polymers, extruded polymers, extruded cellular compositions of the synthetic polymers or as an extruded foam of the synthetic polymer. In a similar manner, the single the material 21 could also be formed of non-synthetic polymers such as silk or rubber in order to obtain desired plasticity and flexibility.

The surface 23 of the endotracheal intubation guide 10 gently engages the inner wall of the airway lumen 15, slidably engage the inner surface of an endotracheal tube and be able to traverse saliva, blood, mucus and other liquids that may obscure the patient's airway. In the present embodiment, the outer surface 23 of the endotracheal intubation guide 10 could be the interface of the single material 21 and the surface that the endotracheal intubation guide 10 is engaging. The single material 21 could be selected for, in addition to its characteristics enabling it to serve as the body 11 and the plastic element 25, for being low-friction and waterproof. In other embodiments outer surface 23 could constitute a thin coating of a distinct material. Forming the outer surface 23 of the endotracheal intubation guide 10 out of a material distinct from the single material 21 could be advantageous as the single material 21 could be exclusively selected for qualities specific to its operation as the body 11 and the plastic element 25.

Looking now to FIG. 2B and FIG. 2C, in another embodiment, the intubation guide is formed to contain a lumen 24 spanning all or substantially all of the body's length. The lumen 24 promotes ventilation during placement of the intubation guide. In one embodiment, the lumen 24 is of sufficient width so as to allow uninterrupted jet ventilation during placement of the endotracheal intubation guide 10.

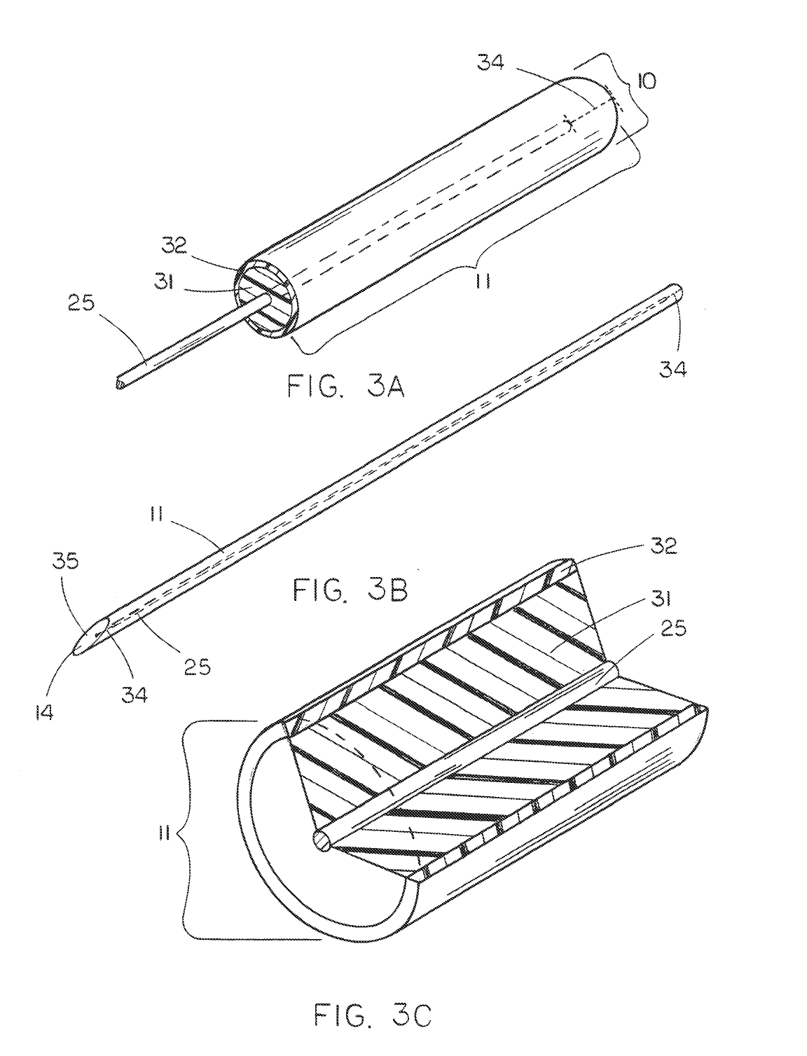

Looking now to FIG. 3A, in some embodiments the endotracheal intubation guide 10 is an assembly of more than one component. The endotracheal intubation guide 10 is formed substantially by a body 11. The body 11 is further formed to receive a plastic element 25. In the present embodiment, the plastic element 25 is a member formed from a malleable material sufficiently plastic to hold an angle bent into it. In some such embodiments, the plastic element 25 is a wire formed of a ductile metal such as steel, aluminum or copper. In other embodiments the plastic element 25 can be any similar flexible member that can undergo plastic deformation. The body 11 of the endotracheal intubation guide 10 is formed to completely surround the plastic element 25. So formed, the endotracheal intubation guide 10 can comprise a plastic element 25 that would, ordinarily, risk substantial trauma to the patient's airway. The body 11, formed of an appropriately soft material can entirely conceal the plastic element 25.

Looking at FIG. 3C, the body 11 is illustrated as being comprised of two components: an inner core 31, which is formed of a soft highly flexible material with minimal elasticity and a thin outer coat 32. The inner core 31 serves to protect the inner wall of the airway lumen 15 from the plastic element 25, to minimize the resistance against the plastic element 25 and maximize the plasticity of the endotracheal intubation guide 10. In a preferred embodiment the inner core 31 is formed of a biocompatible plasticized polyvinylchloride. Other embodiments can utilize similar biocompatible plasticized synthetic polymers in conjunction with other co-polymers. In other embodiments the inner core 31 can be formed of natural polymers such as silk or rubber. The inner core 31 could also be formed of polymerized, plasticized, biocompatible silicon oxides.

The outer coat 32 can be formed entirely by the interface of the inner core 31 and the surfaces engaged by the endotracheal intubation guide 10. The outer coat 32 can also be formed of a distinct material. For example, the outer coat 32 can be formed from a synthetic polymer optimized to be watertight and to have a low coefficient of friction such as a biocompatible thin layer of a plasticized polyvinylchloride resin. In other embodiments the outer coat 32 can be formed of thin layers of similar matter.

Looking now to FIG. 3B, to further protect the patient's airway from the plastic element 25, the inner core 31 is formed to extend beyond the length of the plastic element 25 and create proximal and distal areas 34 comprised primarily of the inner core 31. Furthermore, the distal end 14 of the endotracheal intubation guide 10 can be formed to a different shape so as to facilitate engagement with the glottic opening 62 (not pictured). In some embodiments, the distal end 14 of the endotracheal intubation guide 10 is beveled 35 but in other embodiments the distal end 14 can be tapered, coude or otherwise shaped to facilitate engagement with the glottic opening 62 (not pictured).

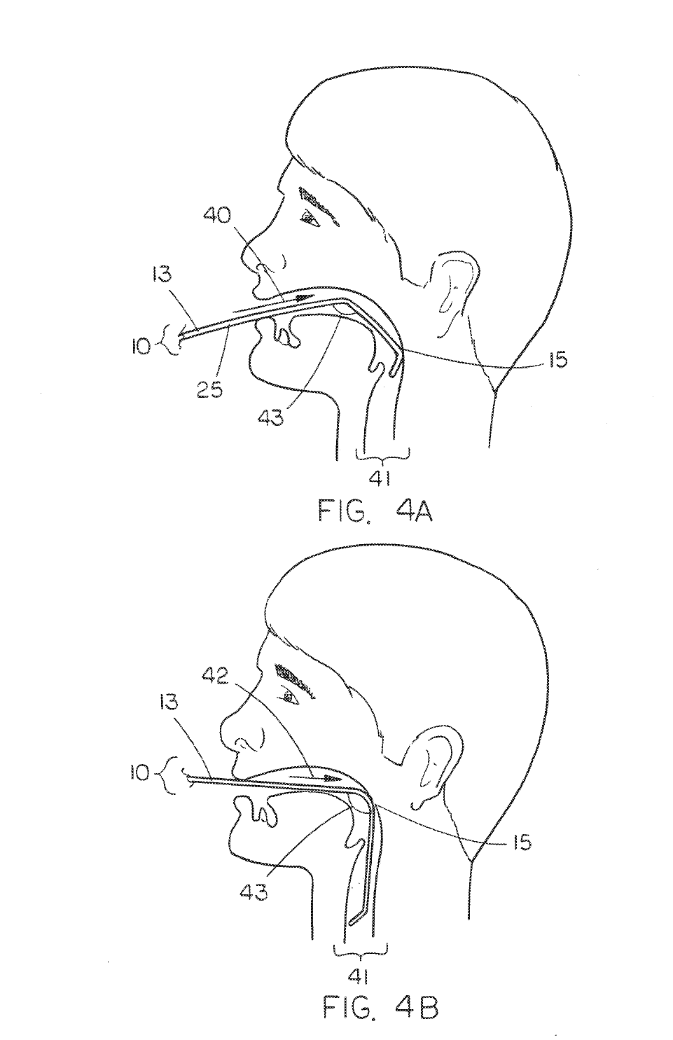

The plastic element 25 may have sufficient give so as to minimize or prevent traumatic engagement with the inner surface of the airway lumen 15. Looking now to FIG. 4A, initial insertion 40 of an endotracheal intubation guide 10, shaped to substantially conform to the patient's endotracheal airway 41, is depicted. In all embodiments, the plastic element 25 of the endotracheal intubation guide 10 has sufficient give so that the engagement with the inner wall of the airway lumen 15 is non-traumatic.

Looking now to FIG. 4B, the continued insertion 42 of the endotracheal intubation guide 10 leads to the deformation of the endotracheal intubation guide 10 as it continues to engage the inner wall of the airway lumen 15. The deformation of the endotracheal intubation guide 10 when engaged with the inner surface of the airway lumen 15 constitutes non-traumatic engagement 43 with the inner surface of the airway lumen 15. The give of the plastic element may generally be sufficient so that the force of initial insertion 40 or continued insertion 42 of the endotracheal intubation guide 10 will cause the endotracheal intubation guide 10 to deform when engaging the inner surface of the airway lumen 15 prior to traumatically engaging the inner surface of the airway lumen 15. For example, in embodiments where the plastic element 25 is a wire formed of ductile metal, the gauge of the wire is thin enough to maximize the amount of give while still retaining plastic deformations so as to practice associated methods of indirect intubation. In embodiments that utilize a steel wire, the plastic element 25 the gauge would be greater than 17 on the Washburn & Moen/U.S. Steel wire gauge scale.

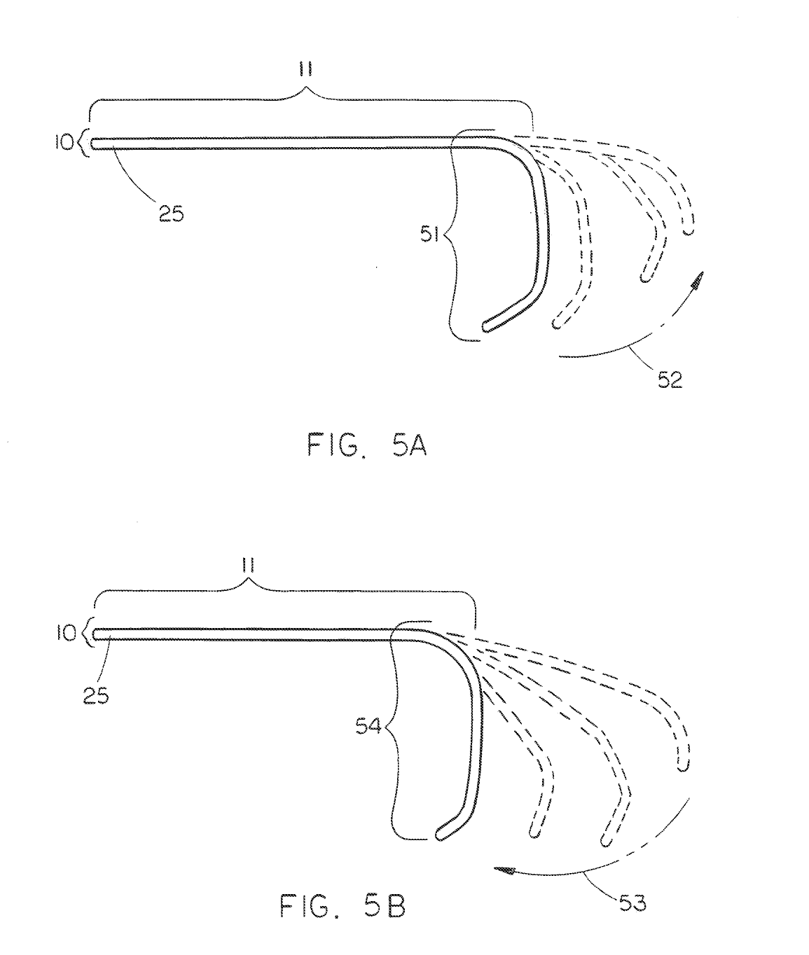

The presently disclosed apparatus further advances the art by granting inexperienced intubators the option of using a pre-formed intubation guide. When presented with a linear, unbent endotracheal intubation guide (e.g. 10 as depicted in FIG. 1A) that is linear and contains no bends, an inexperienced intubator may not know how to shape the intubation guide so as to navigate the patient's airway (e.g. the shaped endotracheal intubation guide 13). FIG. 5A depicts embodiments of the endotracheal intubation guide 10 that are pre-formed to substantially conform to the unaligned shape of the patient's endotracheal airway 41 (not pictured). Moreover, the endotracheal intubation guide 10 can undergo elastic plastic deformation 52 that will return to the preformed shape of the endotracheal intubation guide or a shape that is substantially similar. In one embodiment, the endotracheal intubation guide 10 comprises a soft, malleable and minimally-resistant body 11 and a plastic element 25 that are able to permit elastic-plastic deformations 52. In one embodiment the plastic element 25 could be a member formed from an aluminum alloy selected for its ability to undergo elastic-plastic deformations. In other embodiments the plastic element 25 could be formed in part by the material substantially comprising the body 11 of the endotracheal intubation guide 10.

FIG. 5B depicts the endotracheal intubation guide 10, having undergone elastic-plastic deformation 52 returning 53 to a deformed shape 54 the angles comprising the endotracheal intubation guide 10 are biased towards the preformed shape 51 but are not exactly the same. In a preferred embodiment the pre-formed angles are maintained by a preformed plastic element 25 that can undergo elastic-plastic deformation 53. Depending on the skill of the intubator, and the intubator's associated experience using the endotracheal intubation guide 10, the degree of elasticity is variable. For inexperienced intubators, the amount of elasticity would be relatively high so that the plastic element 25 will bias deformations 52 towards the pre-formed shape 51 of the endotracheal intubation guide 10. Other embodiments would comprise a plastic element 25 that is less elastic so that experienced intubators could more readily deform and shape the endotracheal intubation guide 10. In some embodiments the plastic element 25 would have minimal or no elasticity so as to enable the intubator to shape the endotracheal intubation guide 10 to fit the patient's airway according to his or her judgment.

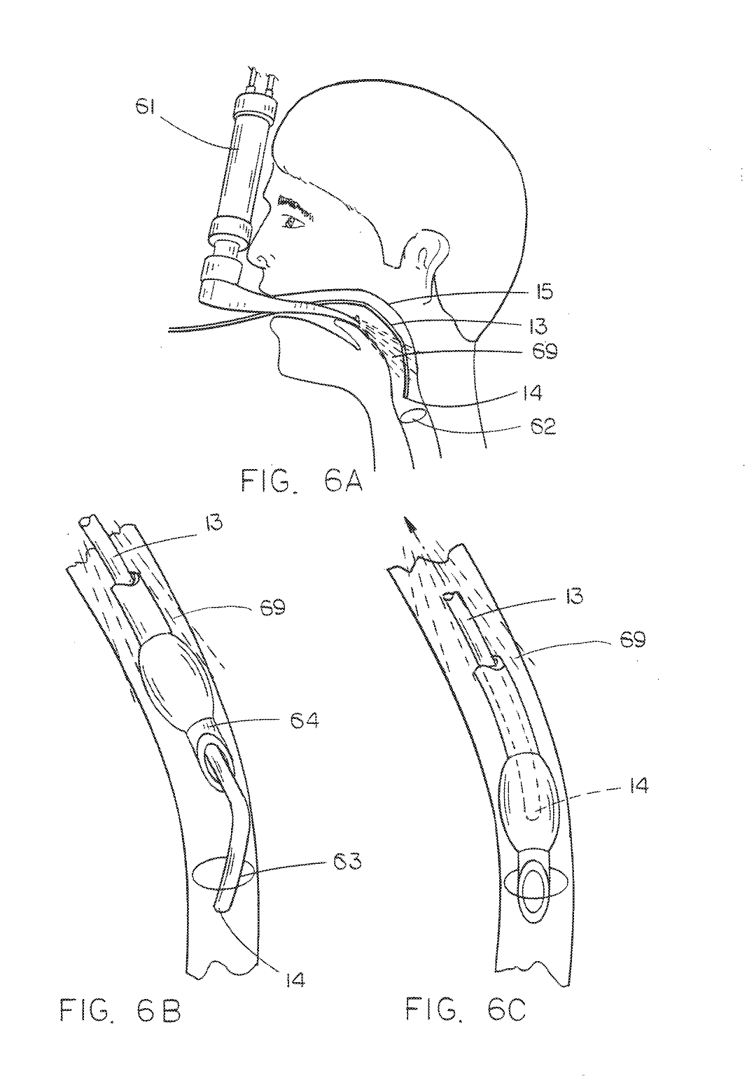

Looking now to FIG. 6A, in another embodiment, the present disclosure describes a method for positioning devices in the patient's airway utilizing an indirect visualization instrument 61. In some embodiments, the indirect visualization instrument is a Stortz video laryngoscope. The method involves obtaining a view of the glottic opening 62 utilizing the indirect visualization mode to see around anatomic obstructions. The intubator then places the shaped endotracheal intubation guide 13 so as to enable the placement of the distal end 14 around anatomical obstructions and into the field of view 69 of the indirect visualization instrument 61. In another embodiment, the shaped endotracheal intubation guide 13 is pre-formed to substantially conform to the general shape of the endotracheal airway.

The intubator need not have the patient's airway 41 (not pictured) aligned so as to enable direct visualization of the glottic opening 62 (not pictured) using a indirect visualization instrument. In general, the first step of intubation is alignment of the patient's airway. Although the definition of a difficult airway is outcome-based, the factors influencing that outcome are often difficulties associated with anatomical obstructions to the alignment of the patient's airway. The practice of the present techniques may allow for successful intubation even when the patient's airway can be less than optimally aligned or cannot be aligned at all.

Looking to FIG. 6B and FIG. 6C, once the distal end 14 of the shaped endotracheal intubation guide 10 is in the field of view 69 of the indirect visualization instrument 61, the intubator can then confirm placement of the intubation guide 63 by indirect visualization. Upon confirmation of placement 63, the intubator can then slide an endotracheal tube 64 over the intubation guide. By maintaining the position of the indirect visualization instrument the intubator can subsequently confirm the placement of the endotracheal tube 64 prior to removing the shaped endotracheal intubation guide 13. Placement of both the shaped endotracheal intubation guide 13 and the endotracheal tube 64 can be performed with minimal alignment of the patient's airway or even in an unaligned airway 17.

In other embodiments the method can utilize other means of indirect visualization. Other video laryngoscopes, such as the glidescope, can be used to indirectly visualize the glottic opening 62. Alternatively, a video laryngeal mask could be used to visualize the glottic opening 62. Even devices such as video stylets or fiber optic bougies could be used as indirect visualization instruments to confirm placement of the intubation guide and endotracheal tube. In some embodiments, however, the indirect visualization instrument provides a high-quality image and an abundant light source.

By way of example, samples of existing intubation guides were tested for tensile strength and compared to that of an intubation guide made according to the present disclosure. The first intubation guides tested were stylets. Stylets may be placed in an intubation tube to pre-form the intubation tube and then be inserted with the intubation tube as a single unit. Because most, if not all, of the stylet will be within the intubation tube during intubation, the interaction and with and possible trauma to the tracheal lumen is not a high concern in the design of stylets. However, maintaining conformity of the intubation tube shape is important so less ductile materials may be used than with bougies. The less ductile materials will often have a higher tensile strength and greater rigidity.

Also tested was an existing bougie. Bougies are used in difficult intubations where the glottic opening cannot be visualized. A coude shaped end may be used as a “feeler” that bounces along the tracheal ridges to confirm placement. Once placed, the bougie is used as a guide and the intubation tube is run over it and into the patient's trachea. The bougie is then removed from the intubation tube.

Because the bougie is used as a guide for patients with difficult airways, bougies are generally made sufficiently resilient to deform the shape of the intubation tube as it is placed over the bougie. However, because the bougie comes in direct contact with the inner surface of the airway during placement, typically more supple materials are used to avoid trauma to the patient's airway.

The existing stylets tested yielded a maximum load of 172-201 lbs. Such relatively high maximum loads are reflective of the design required for a stylet to adequately pre-form an intubation tube.

In contrast, an existing 12 fr (4mm) bougie was tested and yielded a maximum load of about 80 lbs., significantly less than the 12 fr stylet tested.

A bougie made according to the disclosure provided herein, is the BOEDEKER BOUGIE available from Truer Medical Inc., of Orange, Calif. This 12 fr bougie comprises a malleable metal wire core with a low density polyethylene frosted, low friction sheath. The design has been found effective for providing an intubation tube guide while avoiding trauma to a patient's trachea. A segment of this bougie yielded a maximum load of 129 (+/−10) lbs. This tensile strength has shown to lead to a sufficiently resilient apparatus that can function as an intubation tube guide, while maintaining enough give, in combination with the sheath, to avoid trauma to the trachea of most patients.

Although the subject matter has been described in language specific to structural features and/or process operations, it is to be understood that the subject matter defined in the appended claims is not necessarily limited to the specific features or acts described above. Rather, the specific features and acts described above are disclosed as example forms of implementing the claims.

What is claimed is:

1. An apparatus to guide the placement of an endotracheal tube comprising:
a plastic element configured to plastically deform and to at least substantially retain a plastic deformation when the apparatus is placed in minimal engagement with an inner surface of an airway lumen to avoid traumatic engagement with the inner surface, and
a non-rigid body at least partially encapsulating the plastic element, the non-rigid body configured to prevent contact between the plastic member and the inner surface of the airway lumen,
wherein the non-rigid body and the plastic element therein comprise a first end and a distal end, the distal end being configured to be inserted into a field of view of an indirect visualization instrument to facilitate placement of the apparatus in the airway lumen and the first end configured to receive an endotracheal tube for insertion over the apparatus following placement of the apparatus in the airway lumen to guide the endotracheal tube into the airway lumen.

2. The apparatus of claim 1, wherein the non-rigid body is formed of a biocompatible material.

3. The apparatus of claim 2, wherein the biocompatible material comprises at least one of latex, silicone, polyester, nylon, rubber, or silk.

4. The apparatus of claim 1, wherein the non-rigid body is formed from a synthetic polymer selected for biocompatibility and further comprising one or more plasticizers to promote plasticity.

5. The apparatus of claim 1, wherein the plastic element comprises a wire formed of a ductile metal.

6. The apparatus of claim 5, wherein the wire comprises at least one of steel, copper, or aluminum.

7. The apparatus of claim 1, wherein the surface of the non-rigid body is waterproof and promotes slidable engagement with the endotracheal tube.

8. The apparatus of claim 1, wherein the distal end comprises a coude tip and promotes insertive engagement with the glottic opening.

9. The apparatus of claim 1, wherein the non-rigid body comprises a sheath spanning all or substantially all of the body of the apparatus.

10. The apparatus of claim 1, wherein the plastic element is elastic.

11. A method for endotracheal intubation comprising the steps of:
utilizing an indirect visualization instrument to visualize the airway;
placing a series of plastic deformations into an intubation guide so as to place the distal end of the apparatus into the view of the indirect visualization instrument;
utilizing the indirect visualization instrument to confirm the positioning of the intubation guide in the glottic opening;
sliding an edotracheal tube over the intubation guide and position the endotracheal tube in the glottic opening;
confirming the placement of the endotracheal tube with the indirect visualization instrument; and
withdrawing the intubation guide through the lumen of the endotracheal tube.

12. The method of claim 11, where the indirect visualization instrument is selected from a group comprising: a Stortz DCI Video Laryngoscope or a Glidescope Video Laryngoscope, a generic video laryngoscope, a generic video laryngeal masks or a generic fiber optic bougie.

13. The method of claim 11, wherein the airway is not aligned.

14. An intubation guide configured to guide the placement of an endotracheal tube within an airway lumen of a patient, the intubation guide comprising:
a plastic element configured to plastically deform and to at least substantially retain a plastic deformation when the apparatus is placed in minimal engagement with an inner surface of the airway lumen, the plastic element configured to flex when contacting the inner surface of the airway lumen to avoid traumatic engagement with the inner surface; and
a non-rigid body at least partially encapsulating the plastic element, the non-rigid body configured to prevent contact between the plastic member and the inner surface of the airway lumen;
wherein the non-rigid body and the plastic element therein comprise a first end and a distal end, the distal end configured to be inserted in a field of view of an indirect visualization instrument to facilitate placement of the intubation guide in the airway lumen and the first end configured to receive an endotracheal tube for insertion over the intubation guide following placement of the apparatus in the airway lumen to guide the endotracheal tube into the airway lumen.

15. The intubation guide of claim 14, wherein the non-rigid body is formed from a synthetic polymer selected for biocompatibility and further comprising one or more plasticizers to promote plasticity.

16. The intubation guide of claim 14, wherein the plastic element comprises a wire formed of ductile metal.

17. The intubation guide of claim 14, wherein the surface of the non-rigid body is waterproof and promotes slidable engagement with the endotracheal tube.

18. The intubation guide of claim 14, wherein the distal end promotes insertive engagement with the glottic opening.

19. The intubation guide of claim 14, wherein the plastic element is elastic.
